(19) United States
(12) Patent Application Publication
(10) Pub. No.: US 2015/0047645 A1
(43) Pub. Date: Feb. 19, 2015

(54) Novel Male Condom

(71) Applicant: Kurt S. Jumisco, Grosse Pointe Woods, MI (US)

(72) Inventor: Kurt S. Jumisco, Grosse Pointe Woods, MI (US)

(21) Appl. No.: 13/966,542

(22) Filed: Aug. 14, 2013

(51) Int. Cl. A61F 6/04

(52) U.S. Cl. CPC A61F 6/04; A61F 2006/048
USPC 128/844

(57) ABSTRACT

The present invention provides a human male condom. The human male condom includes an open-ended cylinder, an open-ended cylinder of fabric that covers the exterior surface of the open-ended cylinder, an open-ended receiving collar, a nipple, and a detachable receiving cap. A method of using the human male condom is also provided.

### BACKGROUND OF THE INVENTION

Sexually transmitted diseases and family planning drive a large segment of the population to practice birth control. Condoms are a widely used birth control device that not only provides contraception, but also serves to reduce the spread of sexually transmitted diseases. Condoms have been fabricated from various materials and have been structurally modified to provide increased sexual pleasure. An example of such an enhancement is the ribbed condom, wherein ribs are provided along the length of the condom body to provide additional stimulus to the female. Additionally, materials such as lambskin have been used to form the condom body, in order to provide increased sensitivity to the male. However, these enhancements generally provide only relatively moderate stimulation and are not useful to provide other forms of sexual stimulation.

What is needed is a condom that can provide for an enhanced sexual experience.

### SUMMARY OF THE INVENTION

The present invention provides a human male condom. The human male condom includes an open-ended cylinder, an open-ended cylinder of fabric that covers the exterior surface of the open-ended cylinder, an open-ended receiving collar, a nipple, and a detachable receiving cap. The human male condom can be used in a variety of ways including, for example, (1) a traditional male condom, (2) stimulation of the penis, (3) stimulation of male scrotum, (4) stimulation of the female genitals, and (5) stimulation of female nipples and breasts. The human male condom also (1) helps prevent premature ejaculation by building the penile muscles, (2) improves blood circulation, (3) helps reduce or eliminate minor cases of Peyrones Disease, (4) can be used to perform Kegel exercises to treat male urinary incontinence, (5) can be used to impregnate a female when the detachable cap is removed, (6) can improve prostrate health by extracting prostrate fluid, (7) can be used as a breast milk pump, and (8) can assist in reversing inverted nipples. Further, the male condom will not break like a regular condom, is easy to use, prevents sexually transmitted diseases, is virtually indestructible, and is very economical.

The present invention provides a human male condom. The human male condom includes: including: an open-ended cylinder having a proximal end, a distal end, an interior surface, and an exterior surface, wherein the proximal end has a first annular flange on the interior surface, wherein the distal end has a plurality of external sealing threads thereon; an open-ended cylinder of fabric having a proximal elastic band and a distal elastic band, wherein the open-ended cylinder of fabric covers the exterior surface of the open-ended cylinder, wherein the proximal elastic band is sized to fit snugly around the proximal end of the open-ended cylinder, wherein the distal elastic band is sized to fit snugly around the distal end of the open-ended cylinder; an open-ended receiving collar having a proximal end, a distal end, an interior surface having a plurality of internal sealing threads thereon, and an exterior surface; a nipple having a proximal end removably connected to the distal end of the open-ended receiving collar; a detachable receiving cap having an exterior surface, and interior surface, and a proximal open-end, and a closed distal end, and wherein the proximal end of the detachable receiving cap is configured to frictionally engage the exterior surface of the open-ended receiving collar.

In one embodiment, the proximal end of the open-ended cylinder has a larger diameter than the diameter of distal end of the open-ended cylinder. In one embodiment, the open-ended cylinder of fabric includes a non-woven material. In one embodiment, the non-woven material is a porous material.

In one embodiment, the first annular flange on the internal surface of the open-ended cylinder is directed inward toward the distal end. In one embodiment, the distal elastic band has a smaller diameter than the diameter of the proximal elastic band. In one embodiment, an internal diameter of the proximal end of the open-ended receiving collar is greater than an internal diameter of the distal end of the open-ended receiving collar.

In one embodiment, the internal surface of the proximal end of the open-ended receiving collar has a plurality of protrusions to engage the distal elastic band. In one embodiment, the proximal end of the nipple has a second annular flange configured to engage the internal surface of the distal end of the open-ended receiving collar. In one embodiment, the proximal end of the detachable receiving cap is configured with a pressure fitting to frictionally engage the exterior surface of the open-ended receiving collar.

The present invention provides a human male condom. The human male condom includes: an open-ended cylinder having a proximal end, a distal end, an interior surface, and an exterior surface, wherein the proximal end has a first annular flange on the interior surface that is directed inward toward the distal end, wherein the distal end has a plurality of external sealing threads thereon, wherein the proximal end of the open-ended cylinder has a larger diameter than the diameter of the distal end of the open-ended cylinder; an open-ended cylinder of fabric having a proximal elastic band and a distal elastic band, wherein the open-ended cylinder of fabric covers the exterior surface of the open-ended cylinder, wherein the proximal elastic band is sized to fit snugly around the proximal end of the open-ended cylinder, wherein the distal elastic band is sized to fit snugly around the distal end of the open-ended cylinder, wherein the distal elastic band has a smaller diameter than the diameter of the proximal elastic band, an open-ended receiving collar having a proximal end, a distal end, an interior surface having a plurality of internal sealing threads thereon, and an exterior surface, wherein an internal diameter of the proximal end of the open-ended receiving collar is greater than an internal diameter of the distal end of the open-ended receiving collar, wherein the internal surface of the proximal end of the open-ended receiving collar has a plurality of protrusions to engage the distal elastic band; a nipple having a proximal end removably connected to the distal end of the open-ended receiving collar; a detachable receiving cap having an exterior surface, and interior surface, and a proximal open-end, and a closed distal end, and wherein the proximal end of the detachable receiving cap is configured to frictionally engage the exterior surface of the open-ended receiving collar.

In one embodiment, the open-ended cylinder of fabric includes a non-woven material. In one embodiment, the non-woven material is a porous material. In one embodiment, the proximal end of the nipple has a second annular flange configured to engage the internal surface of the distal end of the open-ended receiving collar. In one embodiment, the proximal end of the detachable receiving cap is configured with a pressure fitting to frictionally engage the exterior surface of the open-ended receiving collar.

The present invention provides a human male condom. The human male condom includes: an open-ended cylinder having a proximal end, a distal end, an interior surface, and an exterior surface, wherein the proximal end has a first annular flange on the interior surface that is directed inward toward the distal end, wherein the distal end has a plurality of external sealing threads thereon, wherein the proximal end of the open-ended cylinder has a larger diameter than the diameter of the distal end of the open-ended cylinder; an open-ended cylinder of fabric having a proximal elastic band and a distal elastic band, wherein the open-ended cylinder of fabric covers the exterior surface of the open-ended cylinder, wherein the proximal elastic band is sized to fit snugly around the proximal end of the open-ended cylinder, wherein the distal elastic band is sized to fit snugly around the distal end of the open-ended cylinder, wherein the distal elastic band has a smaller diameter than the diameter of the proximal elastic band, wherein the open-ended cylinder of fabric includes a non-woven material, an open-ended receiving collar having a proximal end, a distal end, an interior surface having a plurality of internal sealing threads thereon, and an exterior surface, wherein an internal diameter of the proximal end of the open-ended receiving collar is greater than an internal diameter of the distal end of the open-ended receiving collar, wherein the internal surface of the proximal end of the open-ended receiving collar has a plurality of protrusions to engage the distal elastic band; a nipple having a proximal end removably connected to the distal end of the open-ended receiving collar, wherein the proximal end of the nipple has a second annular flange configured to engage the internal surface of the distal end of the open-ended receiving collar; a detachable receiving cap having an exterior surface, and interior surface, and a proximal open-end, and a closed distal end, and wherein the proximal end of the detachable receiving cap is configured with a pressure fitting to frictionally engage the exterior surface of the open-ended receiving collar.

The present invention also provides a method of using a human male condom. The method includes: providing a human male condom including: an open-ended cylinder having a proximal end, a distal end, an interior surface, and an exterior surface, wherein the proximal end has a first annular flange on the interior surface, wherein the distal end has a plurality of external sealing threads thereon; an open-ended cylinder of fabric having a proximal elastic band and a distal elastic band, wherein the open-ended cylinder of fabric covers the exterior surface of the open-ended cylinder, wherein the proximal elastic band is sized to fit snugly around the proximal end of the open-ended cylinder, wherein the distal elastic band is sized to fit snugly around the distal end of the open-ended cylinder; an open-ended receiving collar having a proximal end, a distal end, an interior surface having a plurality of internal sealing threads thereon, and an exterior surface; a nipple having a proximal end removably connected to the distal end of the open-ended receiving collar; a detachable receiving cap having an exterior surface, and interior surface, and a proximal open-end, and a closed distal end, and wherein the proximal end of the detachable receiving cap is configured to frictionally engage the exterior surface of the open-ended receiving collar; placing the human male condom over a user's penis with one hand; reducing the air pressure inside the human male condom by pumping on the open-ended cylinder with a second hand; and having the user fornicate with a second individual.

### BRIEF DESCRIPTION OF THE DRAWINGS

Embodiments of the invention may be best understood by referring to the following description and accompanying drawings, which illustrate such embodiments. In the drawings:

FIG. 1 is a perspective-view drawing illustrating an exemplary human male condom.

FIG. 2 is a side-view drawing illustrating an exemplary human male condom.

FIG. 3 is a top-view drawing illustrating an exemplary human male condom.

FIG. 4 is a bottom-view drawing illustrating an exemplary human male condom.

FIG. 5 is a side-view drawing illustrating an exemplary human male condom without an exemplary detachable receiving cap.

FIG. 6 is a side-view drawing illustrating an exemplary open-ended cylinder covered with an exemplary open-ended cylinder of fabric.

FIG. 7 is a bottom-view drawing illustrating an exemplary open-ended cylinder covered with an exemplary open-ended cylinder of fabric.

FIG. 8 is a side-view drawing illustrating an exemplary open-ended cylinder of fabric.

FIG. 9 is a perspective-view drawing illustrating an exemplary open-ended receiving collar with an exemplary nipple.

FIG. 10 is a bottom-view drawing illustrating an exemplary open-ended receiving collar with an exemplary nipple.

FIG. 11 is a perspective top-view drawing illustrating an exemplary detachable receiving cap.

FIG. 12 is a perspective bottom-view drawing illustrating an exemplary detachable receiving cap.

The drawings are not necessarily to scale. Like numbers used in the figures refer to like components, steps, and the like. However, it will be understood that the use of a number to refer to a component in a given figure is not intended to limit the component in another figure labeled with the same number.

### DETAILED DESCRIPTION OF THE INVENTION

The present invention provides a human male condom. The human male condom includes an open-ended cylinder, an open-ended cylinder of fabric that covers the exterior surface of the open-ended cylinder, an open-ended receiving collar, a nipple, and a detachable receiving cap. The human male condom can be used in a variety of ways including, for example, (1) a traditional male condom, (2) stimulation of the penis, (3) stimulation of male scrotum, (4) stimulation of the female genitals, and (5) stimulation of female nipples and breasts. The human male condom also (1) helps prevent premature ejaculation by building the penile muscles, (2) improves blood circulation, (3) helps reduce or eliminate minor cases of Peyrones Disease, (4) can be used to perform Kegel exercises to treat male urinary incontinence, (5) can be used to impregnate a female when the detachable cap is removed, (6) can improve prostrate health by extracting prostrate fluid, (7) can be used as a breast milk pump, and (8) can assist in reversing inverted nipples. Further, the male condom will not break like a regular condom, is easy to use, prevents sexually transmitted diseases, is virtually indestructible, and is very economical.

The following detailed description includes references to the accompanying drawings, which form a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense, and the scope of the present invention is defined by the appended claims and their equivalents.

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

The referenced items are provided solely for their disclosure prior to the filing date of the present application. Nothing herein is to be construed as an admission that the present invention is not entitled to antedate such material by virtue of prior invention.

Unless otherwise indicated, the words and phrases presented in this document have their ordinary meanings to one of skill in the art. Such ordinary meanings can be obtained by reference to their use in the art and by reference to general and scientific dictionaries, for example, Webster's Third New International Dictionary, Merriam-Webster Inc., Springfield, Mass., 1993 and The American Heritage Dictionary of the English Language, Houghton Mifflin, Boston Mass., 1981.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The following explanations of certain terms are meant to be illustrative rather than exhaustive. These terms have their ordinary meanings given by usage in the art and in addition include the following explanations.

As used herein, the term “about” refers to a variation of 10 percent of the value specified; for example about 50 percent carries a variation from 45 to 55 percent.

As used herein, the term “and/or” refers to any one of the items, any combination of the items, or all of the items with which this term is associated.

As used herein, the singular forms “a,” “an,” and “the” include plural reference unless the context clearly dictates otherwise. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation.

As used herein, the term “coupled” means the joining of two members directly or indirectly to one another. Such joining may be stationary in nature or movable in nature and/or such joining may allow for the flow of fluids, electricity, electrical signals, or other types of signals or communication between two members. Such joining may be achieved with the two members or the two members and any additional intermediate members being integrally formed as a single unitary body with one another or with the two members or the two members and any additional intermediate members being attached to one another. Such joining may be permanent in nature or alternatively may be removable or releasable in nature.

As used herein, the terms “include,” “for example,” “such as,” and the like are used illustratively and are not intended to limit the present invention.

As used herein, the terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the terms “front,” “back,” “rear,” “upper,” “lower,” “right,” and “left” in this description are merely used to identify the various elements as they are oriented in the FIGS, with “front,” “back,” and “rear” being relative apparatus. These terms are not meant to limit the element which they describe, as the various elements may be oriented differently in various applications.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element without departing from the teachings of the disclosure.

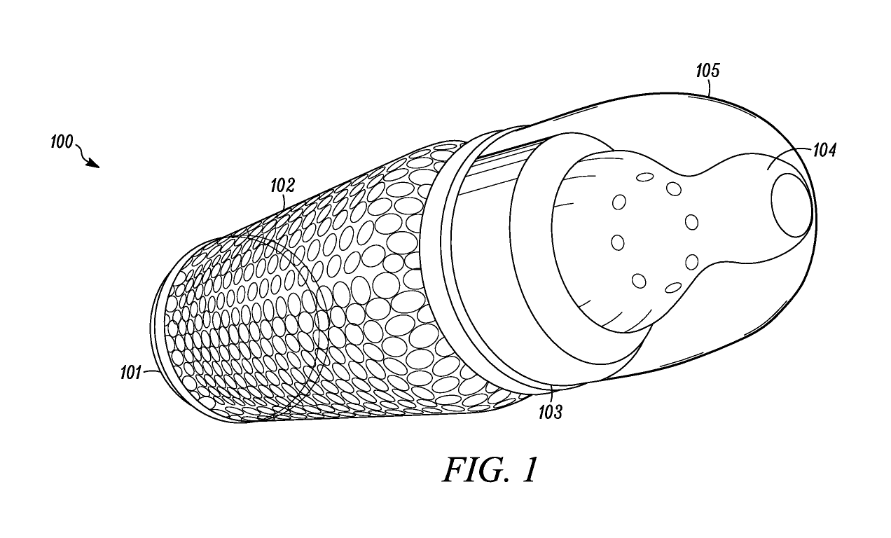

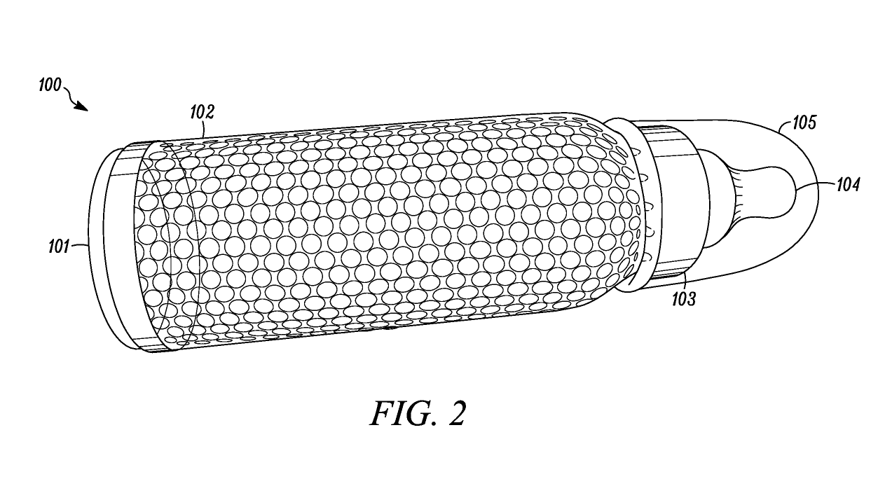

FIGS. 1-2 are a perspective and a side-view drawing illustrating an exemplary human male condom 100. The human male condom 100 includes an open-ended cylinder 101, an open-ended cylinder of fabric 102 that covers the exterior surface of the open-ended cylinder 101, an open-ended receiving collar 103, a nipple 104, and a detachable receiving cap 105.

In one embodiment, the open-ended cylinder 101, open-ended receiving collar 103, and the detachable receiving cap 105 are made from a food-grade or medical-grade polymer. In one embodiment, the open-ended cylinder 101 is made from a flexible food-grade or medical-grade polymer. In one embodiment, the open-ended receiving collar 103 and detachable receiving cap 105 are made from a semi-rigid food-grade or medical-grade polymer. In one embodiment, the open-ended receiving collar 103 and detachable receiving cap 105 are made from a rigid food-grade or medical-grade polymer. In one embodiment, the nipple 104 is made from a food-grade or medical-grade rubber.

In one embodiment, the open-ended cylinder of fabric 102 is made from a woven material. In one embodiment, the open-ended cylinder of fabric 102 is made from a non-woven material. In one embodiment, the woven material is a porous material. In one embodiment, the woven material is a non-porous material. In one embodiment, the non-woven material is a porous material. In one embodiment, the non-woven material is a non-porous material.

To assemble the human male condom 100, the user inserts the open-ended cylinder of fabric 102 over the exterior of the open-ended cylinder 101 so that the wide end of the open-ended cylinder of fabric 102 covers the wide end of the open-ended cylinder 101 and the narrow end of the open-ended cylinder of fabric 102 covers the narrow end of the open-ended cylinder 101. Next, the user installs the nipple 104 into the distal end of the open-ended receiving collar 103 and threads the proximal end of the open-ended receiving collar 103 onto the external sealing threads of the open-ended cylinder 101. The proximal end of the open-ended receiving collar 103 has a series of protrusions (not shown) extending therefrom to engage the distal end of the open-ended cylinder of fabric 102 to hold it in place. Finally, the user attaches the detachable receiving cap 105 to the open-ended receiving collar 103 by applying pressure.

To use the human male condom 100, the user places the human male condom 100 over his penis (not shown) to form a seal and applies pressure repeated to the exterior surface of the open-ended cylinder 101 with his hand. This action reduces the air pressure inside the human male condom 100 so that blood is drawn into the user's penis. After stimulation, the user then inserts the human male condom 100, which contains his penis, into a receptive female partner.

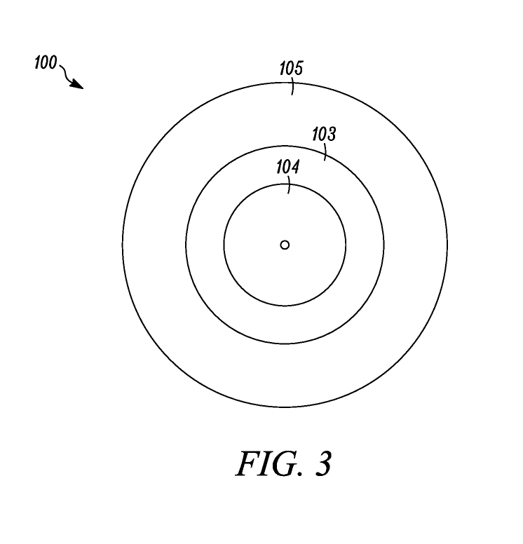

FIG. 3 is a top-view drawing illustrating an exemplary human male condom 100. The human male condom 100 includes an open-ended cylinder (not shown), an open-ended cylinder of fabric (not shown) that covers the exterior surface of the open-ended cylinder (not shown), an open-ended receiving collar 103, a nipple 104, and a detachable receiving cap 105.

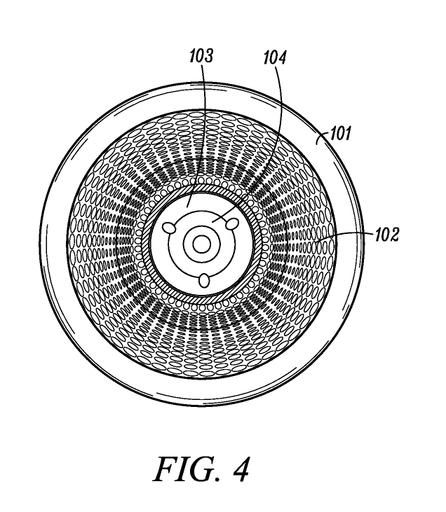

FIG. 4 is a bottom-view drawing illustrating an exemplary human male condom 100. The human male condom 100 includes an open-ended cylinder 101, an open-ended cylinder of fabric 102 that covers the exterior surface of the open-ended cylinder 101, an open-ended receiving collar 103, a nipple 104, and a detachable receiving cap (not shown).

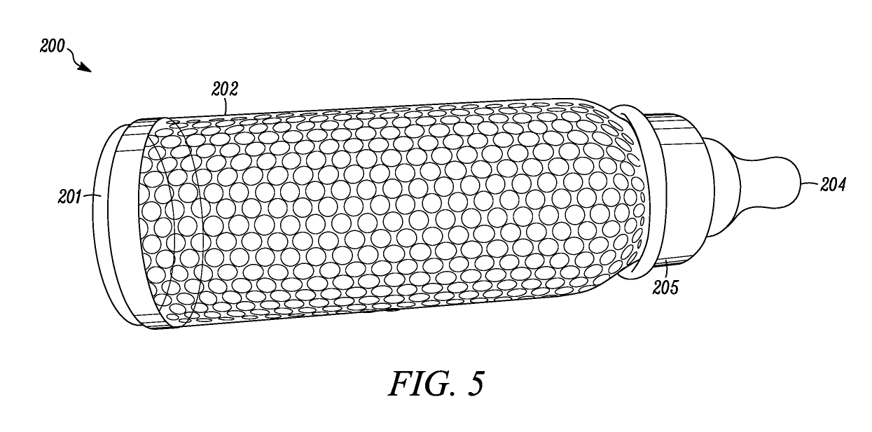

FIG. 5 is a side-view drawing illustrating an exemplary human male condom 200 without an exemplary detachable receiving cap. The exemplary human male condom 200 includes an open-ended cylinder 201, an open-ended cylinder of fabric 202 that covers the exterior surface of the open-ended cylinder 201, an open-ended receiving collar 203, a nipple 204, and a detachable receiving cap (not shown).

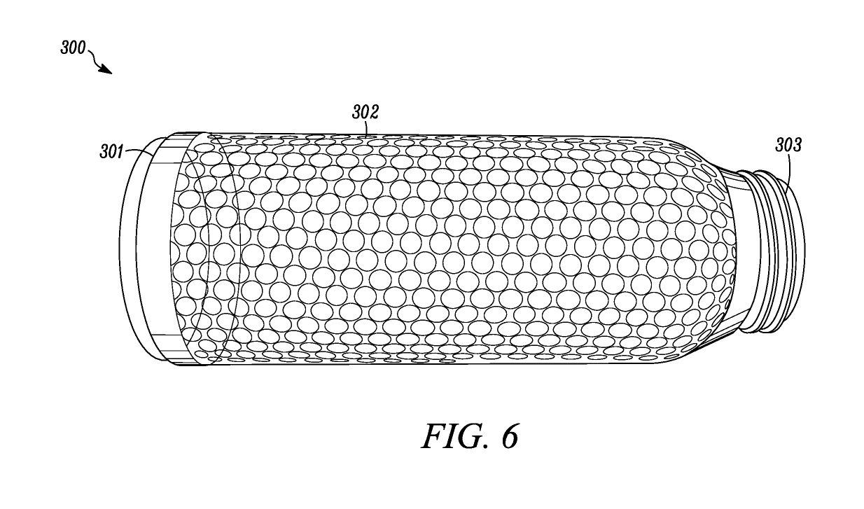

FIG. 6 is a side-view drawing illustrating an exemplary human male condom 300 without an open-ended receiving collar, a nipple, and a detachable receiving cap. The exemplary human male condom 300 includes an open-ended cylinder 301, an open-ended cylinder of fabric 302 that covers the exterior surface of the open-ended cylinder 301, and a series of external threads 303.

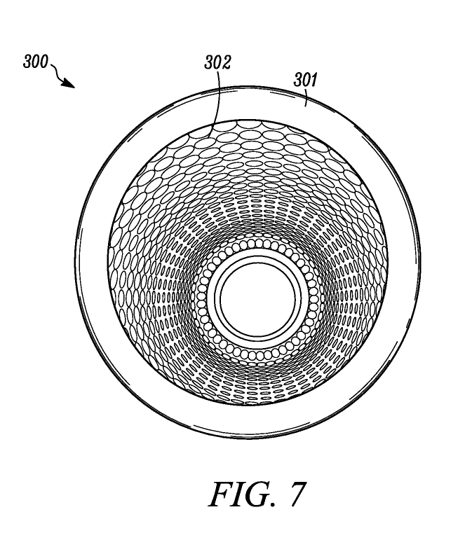

FIG. 7 is a side-view drawing illustrating an exemplary human male condom 300 without an open-ended receiving collar, a nipple, and a detachable receiving cap. The exemplary human male condom 300 includes an open-ended cylinder 301, an open-ended cylinder of fabric 302 that covers the exterior surface of the open-ended cylinder 301, and a series of external threads (not shown).

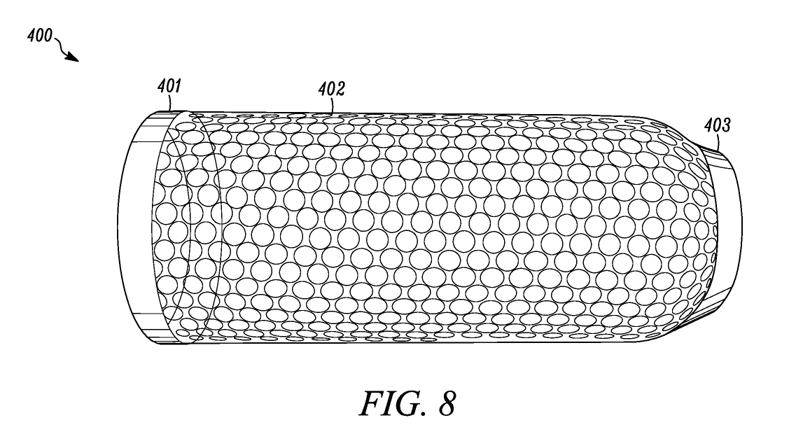

FIG. 8 is a side-view drawing illustrating an exemplary open-ended cylinder of fabric 400. The open-ended cylinder of fabric 400 includes a proximal elastic band 401, fabric 402, and a distal elastic band 403. In one embodiment, the proximal band 402 of the open-ended fabric 400 has a larger diameter than the diameter distal band 403 of the open-ended fabric 400. In another embodiment, the proximal band 402 of the open-ended fabric 400 has a diameter that is equal to the diameter of the distal band 403 of the open-ended fabric 400. In yet another embodiment, the proximal band 402 of the open-ended fabric 400 has a smaller diameter than the diameter of distal band 403 of the open-ended fabric 400. The proximal elastic band 401, fabric 402, and a distal elastic band 403 each independently fit tightly around the exterior of the open-ended cylinder (not shown).

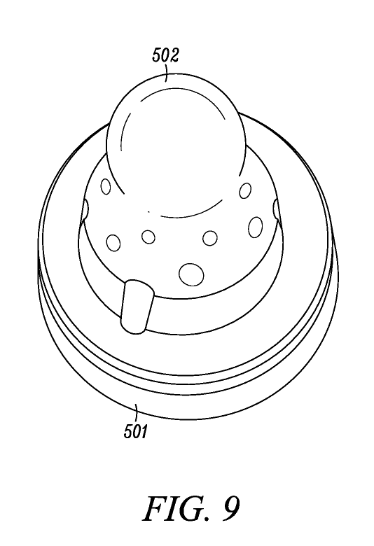

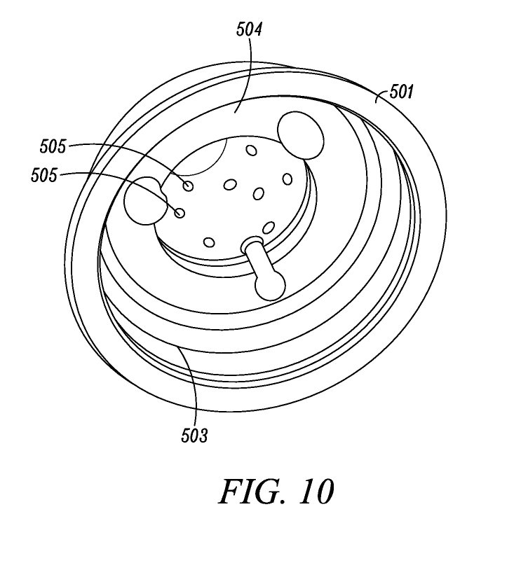

FIGS. 9-10 are various drawings illustrating an exemplary open-ended receiving collar 501 with an exemplary nipple 502. The open-ended receiving collar 501 includes internal sealing threads 503 on the interior surface. The internal sealing threads 503 are spaced to tightly engage the external sealing threads 305 on the open-ended cylinder 301, as shown in FIGS. 6-7. The proximal end of the nipple includes annular flange 504 that engages the distal end of the open-ended receiving collar 501. The distal end of the nipple 502 also includes a plurality of small holes 505.

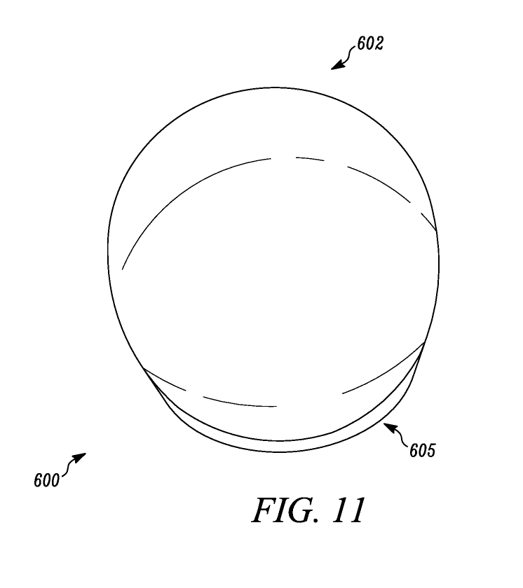

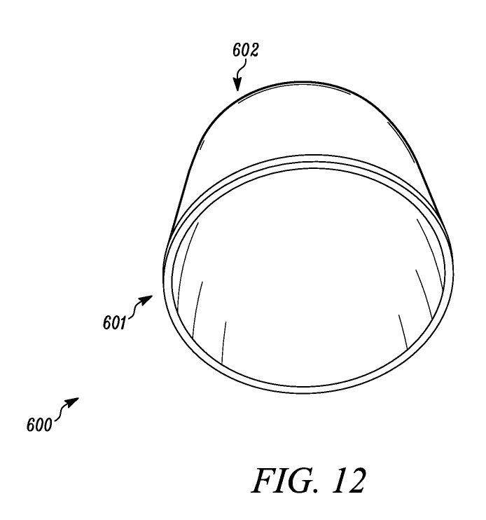

FIGS. 11-12 are various drawings illustrating an exemplary detachable receiving cap 600. The detachable receiving cap 600 has a proximal end 601 and a distal end 602. The proximal end 601 of the detachable receiving cap 600 is configured to fit the distal end of the open-ended receiving collar (not shown).

In the claims provided herein, the steps specified to be taken in a claimed method or process may be carried out in any order without departing from the principles of the invention, except when a temporal or operational sequence is explicitly defined by claim language. Recitation in a claim to the effect that first a step is performed then several other steps are performed shall be taken to mean that the first step is performed before any of the other steps, but the other steps may be performed in any sequence unless a sequence is further specified within the other steps. For example, claim elements that recite “first A, then B, C, and D, and lastly E” shall be construed to mean step A must be first, step E must be last, but steps B, C, and D may be carried out in any sequence between steps A and E and the process of that sequence will still fall within the four corners of the claim.

Furthermore, in the claims provided herein, specified steps may be carried out concurrently unless explicit claim language requires that they be carried out separately or as parts of different processing operations. For example, a claimed step of doing X and a claimed step of doing Y may be conducted simultaneously within a single operation, and the resulting process will be covered by the claim. Thus, a step of doing X, a step of doing Y, and a step of doing Z may be conducted simultaneously within a single process step, or in two separate process steps, or in three separate process steps, and that process will still fall within the four corners of a claim that recites those three steps.

Similarly, except as explicitly required by claim language, a single substance or component may meet more than a single functional requirement, provided that the single substance or component fulfills the more than one functional requirement as specified by claim language.

All patents, patent applications, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Additionally, all claims in this application, and all priority applications, including but not limited to original claims, are hereby incorporated in their entirety into, and form a part of, the written description of the invention.

Applicant reserves the right to physically incorporate into this specification any and all materials and information from any such patents, applications, publications, scientific articles, web sites, electronically available information, and other referenced materials or documents. Applicant reserves the right to physically incorporate into any part of this document, including any part of the written description, the claims referred to above including but not limited to any original claims.

What is claimed is:

1. A human male condom comprising:
an open-ended cylinder having a proximal end, a distal end, an interior surface, and an exterior surface,
wherein the proximal end has a first annular flange on the interior surface,
wherein the distal end has a plurality of external sealing threads thereon;
an open-ended cylinder of fabric having a proximal elastic band and a distal elastic band,
wherein the open-ended cylinder of fabric covers the exterior surface of the open-ended cylinder,
wherein the proximal elastic band is sized to fit snugly around the proximal end of the open-ended cylinder,
wherein the distal elastic band is sized to fit snugly around the distal end of the open-ended cylinder;
an open-ended receiving collar having a proximal end, a distal end, an interior surface having a plurality of internal sealing threads thereon, and an exterior surface;
a nipple having a proximal end removably connected to the distal end of the open-ended receiving collar;
a detachable receiving cap having an exterior surface, and interior surface, and a proximal open-end, and a closed distal end, and
wherein the proximal end of the detachable receiving cap is configured to frictionally engage the exterior surface of the open-ended receiving collar.

2. The human male condom of claim 1, wherein the proximal end of the open-ended cylinder has a larger diameter than the diameter of distal end of the open-ended cylinder.

3. The human male condom of claim 1, wherein the open-ended cylinder of fabric comprises a non-woven material.

4. The human male condom of claim 3, wherein the non-woven material is a porous material.

5. The human male condom of claim 1, wherein the first annular flange on the internal surface of the open-ended cylinder is directed inward toward the distal end.

6. The human male condom of claim 1, wherein the distal elastic band has a smaller diameter than the diameter of the proximal elastic band.

7. The human male condom of claim 1, wherein an internal diameter of the proximal end of the open-ended receiving collar is greater than an internal diameter of the distal end of the open-ended receiving collar.

8. The human male condom of claim 7, wherein the internal surface on the proximal end of the open-ended receiving collar has a plurality of protrusions to engage the distal elastic band.

9. The human male condom of claim 1, wherein the proximal end of the nipple has a second annular flange configured to engage the internal surface on the distal end of the open-ended receiving collar.

10. The human male condom of claim 1, wherein the proximal end of the detachable receiving cap is configured with a pressure fitting to frictionally engage the exterior surface of the open-ended receiving collar.

11. A human male condom comprising:
an open-ended cylinder having a proximal end, a distal end, an interior surface, and an exterior surface,
wherein the proximal end has a first annular flange on the interior surface that is directed inward toward the distal end,
wherein the distal end has a plurality of external sealing threads thereon,
wherein the proximal end of the open-ended cylinder has a larger diameter than the diameter of the distal end of the open-ended cylinder;
an open-ended cylinder of fabric having a proximal elastic band and a distal elastic band,
wherein the open-ended cylinder of fabric covers the exterior surface of the open-ended cylinder,
wherein the proximal elastic band is sized to fit snugly around the proximal end of the open-ended cylinder,
wherein the distal elastic band is sized to fit snugly around the distal end of the open-ended cylinder,
wherein the distal elastic band has a smaller diameter than the diameter of the proximal elastic band,
an open-ended receiving collar having a proximal end, a distal end, an interior surface having a plurality of internal sealing threads thereon, and an exterior surface,
wherein an internal diameter of the proximal end of the open-ended receiving collar is greater than an internal diameter of the distal end of the open-ended receiving collar,
wherein the internal surface of the proximal end of the open-ended receiving collar has a plurality of protrusions to engage the distal elastic band;
a nipple having a proximal end removably connected to the distal end of the open-ended receiving collar;
a detachable receiving cap having an exterior surface, and interior surface, and a proximal open-end, and a closed distal end, and
wherein the proximal end of the detachable receiving cap is configured to frictionally engage the exterior surface of the open-ended receiving collar.

12. The human male condom of claim 11, wherein the open-ended cylinder of fabric comprises a non-woven material.

13. The human male condom of claim 12, wherein the non-woven material is a porous material.

14. The human male condom of claim 11, wherein the proximal end of the nipple has a second annular flange configured to engage the internal surface of the distal end of the open-ended receiving collar.

15. The human male condom of claim 11, wherein the proximal end of the detachable receiving cap is configured with a pressure fitting to frictionally engage the exterior surface of the open-ended receiving collar.

16. A human male condom comprising:
an open-ended cylinder having a proximal end, a distal end, an interior surface, and an exterior surface,
wherein the proximal end has a first annular flange on the interior surface that is directed inward toward the distal end,
wherein the distal end has a plurality of external sealing threads thereon,
wherein the proximal end of the open-ended cylinder has a larger diameter than the diameter of the distal end of the open-ended cylinder;
an open-ended cylinder of fabric having a proximal elastic band and a distal elastic band,
wherein the open-ended cylinder of fabric covers the exterior surface of the open-ended cylinder,
wherein the proximal elastic band is sized to fit snugly around the proximal end of the open-ended cylinder,
wherein the distal elastic band is sized to fit snugly around the distal end of the open-ended cylinder,
wherein the distal elastic band has a smaller diameter than the diameter of the proximal elastic band,
wherein the open-ended cylinder of fabric comprises a non-woven material,
an open-ended receiving collar having a proximal end, a distal end, an interior surface having a plurality of internal sealing threads thereon, and an exterior surface,
wherein an internal diameter of the proximal end of the open-ended receiving collar is greater than an internal diameter of the distal end of the open-ended receiving collar,
wherein the internal surface of the proximal end of the open-ended receiving collar has a plurality of protrusions to engage the distal elastic band;
a nipple having a proximal end removably connected to the distal end of the open-ended receiving collar,
wherein the proximal end of the nipple has a second annular flange configured to engage the internal surface of the distal end of the open-ended receiving collar;
a detachable receiving cap having an exterior surface, and interior surface, and a proximal open-end, and a closed distal end, and
wherein the proximal end of the detachable receiving cap is configured with a pressure fitting to frictionally engage the exterior surface of the open-ended receiving collar.
